(19) United States
(12) Patent Application Publication
(10) Pub. No.: US 2025/0214979 A1
(43) Pub. Date: Jul. 3, 2025

(54) COMPOUNDS FOR TREATING PSYCHOSTIMULANT MISUSE

(71) Applicant: University of Florida Research Foundation, Incorporated, Gainesville, FL (US)

(72) Inventors: Christopher R. McCurdy, Gainesville, FL (US); Sarah Mallory Burns, Gainesville, FL (US); Shelby Tyler Green, Gainesville, FL (US)

(73) Assignee: University of Florida Research Foundation, Incorporated, Gainesville, FL (US)

(21) Appl. No.: 18/851,996

(22) PCT Filed: Mar. 30, 2023
(86) PCT No.: PCT/US2023/016817

§ 371 Date: Sep. 27, 2024

Related U.S. Application Data
(60) Provisional application No. 63/325,513, filed on Mar. 30, 2022.

(51) Int. Cl. C07D 413/06; A61K 31/553; A61K 31/554; C07D 403/06; C07D 417/06; C07D 491/107

(52) U.S. Cl. CPC C07D 413/06; A61K 31/553; A61K 31/554; C07D 403/06; C07D 417/06; C07D 491/107

(57) ABSTRACT

Described herein are new dual sigma receptor and dopamine transporter small molecule antagonists. As these compounds are antagonists of both sigma receptors and dopamine transporters, these compounds dose-dependently antagonize stimulant self-administration and also diminish the effects of stimulant effects on dopamine level, thus indicating minimal abuse liability.

### RELATED APPLICATIONS

The present application claims priority under 35 U.S.C. § 119(e) to U.S. provisional application, U.S. Ser. No. 63/325,513, filed Mar. 30, 2022, which is incorporated herein by reference.

### BACKGROUND

Sigma receptors are a well-defined unique class of receptors and are highly expressed in the central nervous system and also widely distributed in peripheral organs and tissues that serve as targets for psychostimulant drugs (Matsumoto, R. et al. Expert Rev. Clin. Pharmacol. 2009; 2:351-358). Many commonly abused drugs interact with sigma receptors including, for example, cocaine, methamphetamine, and even some opioids (Matsumoto, R. et al. Expert Rev. Clin. Pharmacol. 2009; 2:351-358). In addition to sigma receptors being associated with psychostimulant drugs, these receptors are also associated with various other diseases and disorders including depression, anxiety, schizophrenia, psychosis, pain, Alzheimer's disease, Parkinson's disease, amyotrophic lateral sclerosis, retinal diseases, and stroke (Hayashi, T. et al. CNS Drugs 2004; 18, 269-284; Hayashi, T. et al. Expert Opin. Ther. Targets 2011; 15, 557-577; Kaushal, N. et al. Eur. Neuropsychopharmacol. 2013; 23, 960-971). Additionally, sigma receptors play a role in cancer (Aydar, R. et al. Cancer Res. 2004; 64, 5029-5035; Van Waarde, A. et al. Curr. Pharm. Des. 2010; 16, 3519-3537), cardiovascular diseases (Monassier, L. et al. Fundam. Clin. Pharmacol. 2002; 16, 1-8), inflammatory and autoimmune diseases (Bourrie, B. et al. Expert Opin. Investig. Drugs 2004; 5, 1158-1163; Su, T. P. et al. Science, 1988; 240, 219-221; Wolfe, S. A. et al. J. Pharmacol. Exp. Ther. 1988; 247, 1114-1119).

Although sigma receptors remain a target for developing pharmaceuticals for treating stimulant abuse as sigma-receptor antagonists block many effects of stimulants, sigma receptors do not block reinforcing effects assessed with self-administration. However, studies have suggested that the dopamine transporter is a viable target for attenuating the reinforcing effects of stimulants (Ritz, M. C. et al. Science 1987; 237, 1219-1223; Kuhar, M. J. et al. Trends Neurosci. 1991; 14, 299-302). Thus, pharmaceuticals with a dual effect on both dopamine transporter and sigma receptors are proposed to decrease self-administration, resulting treating stimulant abuse, while at the same time presenting a low liability of abuse. Thus, a dual targeting approach aimed at simultaneous interactions at sigma receptors and dopamine transporters has been explored (Hiranita, T. et al. J. Pharmacol. Exp. Ther. 2011; 339(2): 662-77).

Unfortunately, even with these developments in pharmaceutical design, there are no approved medications to treat stimulant abuse or addiction, even though this problem continues to expand. For example, in the United States alone, drug-involved overdose deaths increased by about 30% from 2019 to 2020 (National Institute on Drug Abuse, Overdose Death Rates, https://nida.nih.gov/drug-topics/trends-statistics/overdose-death-rates). Thus, there remains a need to develop pharmaceuticals capable of treating stimulate abuse and/or addiction.

### SUMMARY OF THE INVENTION

Described herein are new dual sigma receptor and dopamine transporter small molecule antagonists. As these compounds are antagonists of both sigma receptors and dopamine transporters, these compounds dose-dependently antagonize stimulant self-administration and also diminish the effects of stimulant effects on dopamine level, thus indicating minimal abuse liability. These compounds also present suitable metabolic stability and bioavailability.

Thus, in one aspect, provided herein are compounds of Formula (I):

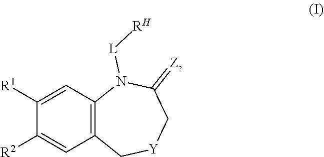

or pharmaceutically acceptable salt thereof, wherein R¹, R², L, R^{H}, Y, and Z are defined herein. In some embodiments, R^{H} is

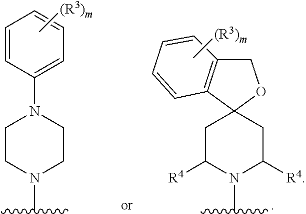

In certain embodiments, the compound of Formula (I) is of the formula:

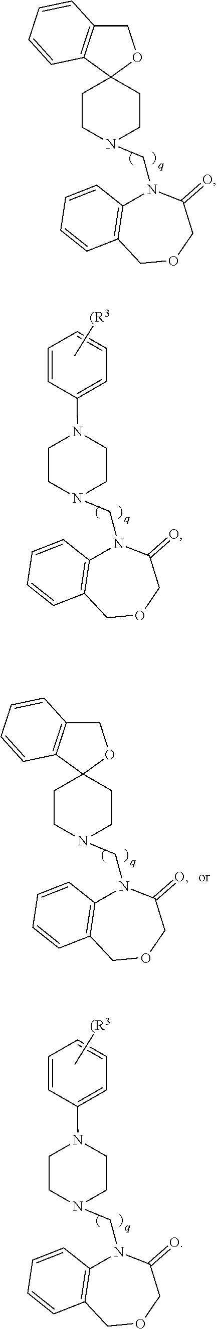

In some embodiments, the compound of Formula I is:

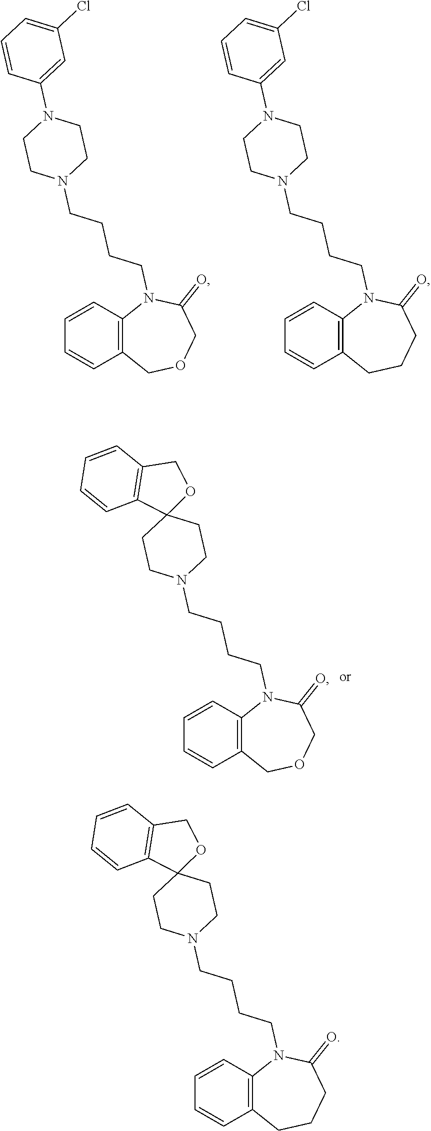 [SMILES: O=C1COCc2ccccc2N1CCCCN1CCN(c2cccc(Cl)c2)CC1 ; O=C1CCCc2ccccc2N1CCCCN1CCN(c2cccc(Cl)c2)CC1 ; O=C1COCc2ccccc2N1CCCCN1CCC2(CC1)OCc1ccccc12 ; O=C1CCCc2ccccc2N1CCCCN1CCC2(CC1)OCc1ccccc12]

In another aspect, provided herein are compositions comprising a compound provided herein (e.g., a compound of Formula (I)), or pharmaceutically acceptable salt thereof, and a pharmaceutically acceptable excipient

In an additional aspect, the present disclosure provides methods of treating or preventing substance intake by a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)).

In another aspect, provided herein are methods of treating or preventing a substance use disorder in a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)).

In one aspect, the present disclosure provides methods of treating the symptoms of substance use disorder in a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)).

In an additional aspect, the present disclosure provides methods of treating or preventing substance addiction by a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)).

In another aspects, provided herein are methods of treating the symptoms of substance addiction by a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)).

In one aspect, the present disclosure provides methods of treating or preventing neurotoxic effects resulting from substance use disorder, substance addiction, and/or substance intake in a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)).

In a further aspect, provided herein are methods of treating or preventing a disease or disorder associated with one or more sigma receptors comprising administering to a subject a therapeutically effective amount of a compound provided herein (e.g., a compound of Formula (I)), or pharmaceutically acceptable salt thereof, or a composition provided herein (e.g., a composition comprising a compound of Formula (I)).

In an additional aspect, the present disclosure provides methods of treating or preventing a disease or disorder associated with one or more dopamine transporters comprising administering to a subject a therapeutically effective amount of a compound provided herein (e.g., a compound of Formula (I)), or pharmaceutically acceptable salt thereof, or a composition provided herein (e.g., a composition comprising a compound of Formula (I)).

In another aspects, provided herein are methods of treating or preventing a disease or disorder associated with one or more dopamine transporters and with one or more sigma receptors comprising administering to a subject a therapeutically effective amount of a compound provided herein (e.g., a compound of Formula (I)), or pharmaceutically acceptable salt thereof, or a composition provided herein (e.g., a composition comprising a compound of Formula (I)).

In one aspect, the present disclosure provides a kit comprising: a compound provided herein (e.g., a compound of Formula (I)), or pharmaceutically acceptable salt thereof, or a composition provided herein (e.g., a composition comprising a compound of Formula (I)); and instructions for administering the compound, or pharmaceutically acceptable salt thereof, or composition to a subject.

Additional aspects of any of the above methods are those wherein the subject is in need of such treatment, and those wherein the subject is identified as in need of such treatment.

The details of certain embodiments of the disclosure are set forth in the Detailed Description of Certain Embodiments, as described below. Other features, objects, and advantages of the disclosure will be apparent from the Definitions, Figures, Examples, and Claims. It should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The following drawings provide non-limiting examples of the invention.

FIG. 1 shows a histogram for sigma 1 receptor inhibition with various compounds of the disclosure and CM699 as a comparative compound.

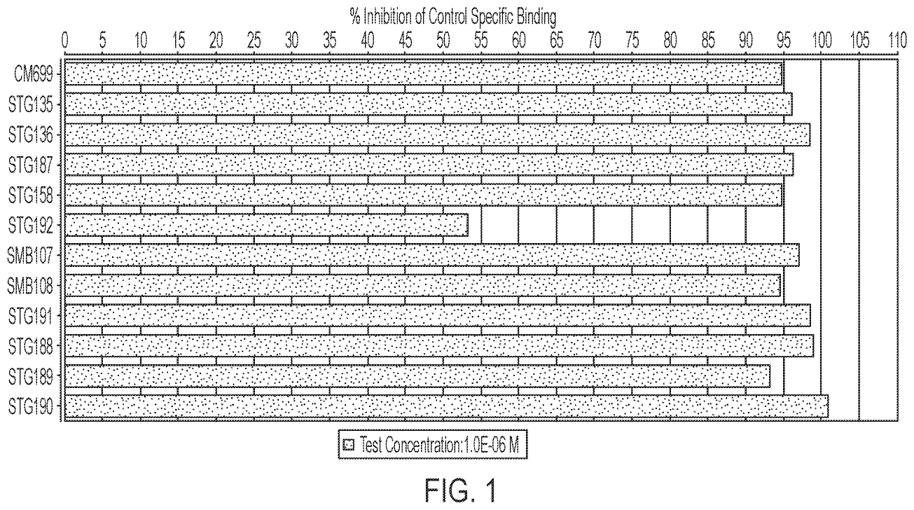

FIG. 2 shows a histogram for sigma 2 receptor inhibition with various compounds of the disclosure and CM699 as a comparative compound.

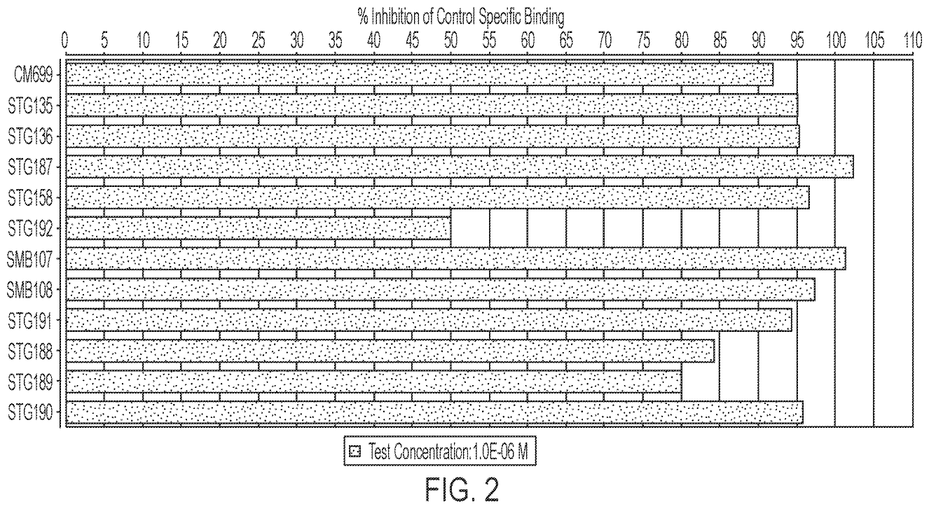

FIG. 3 shows a histogram for dopamine transporter inhibition with various compounds of the disclosure and CM699 as a comparative compound.

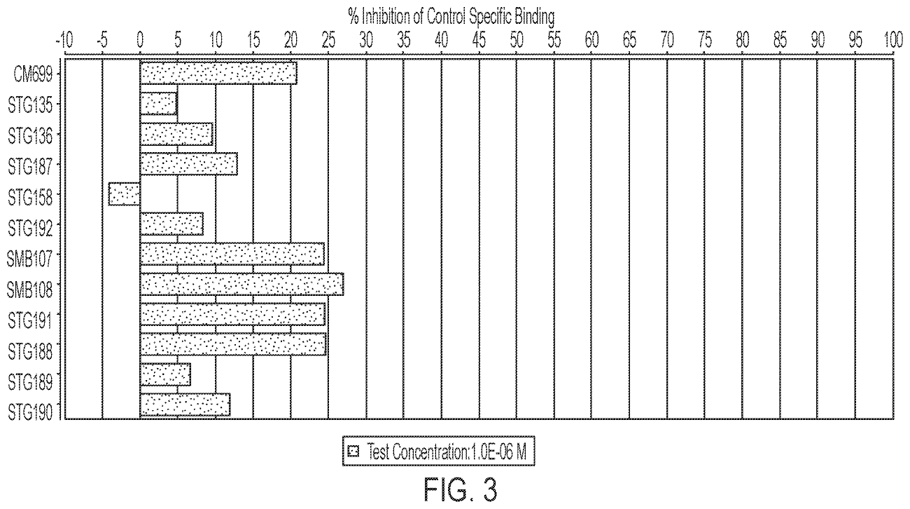

### DEFINITIONS

For convenience, certain terms employed herein, in the specification, examples and appended claims are collected herein.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds and oligonucleotides described herein, even though only a single tautomeric form may be represented. All such isomeric forms of such compounds herein are expressly included in the present disclosure. The term “isomers” is intended to include diastereoisomers, enantiomers, regioisomers, structural isomers, rotational isomers, tautomers, and the like. For compounds that contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

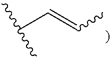

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group-OH. The term “substituted hydroxyl” or “substituted hydroxyl,” by extension, refers to a hydroxyl group wherein the oxygen atom directly attached to the parent molecule is substituted with a group other than hydrogen.

The term “thiol” or “thio” refers to the group-SH. The term “substituted thiol” or “substituted thio,” by extension, refers to a thiol group wherein the sulfur atom directly attached to the parent molecule is substituted with a group other than hydrogen.

The term “amino” refers to the group-NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen. The term “disubstituted amino” refers to an amino group wherein the nitrogen atom directly attached to the parent molecule is substituted with two groups other than hydrogen. The term “trisubstituted amino” refers to an amino group wherein the nitrogen atom directly attached to the parent molecule is substituted with three non-hydrogen groups.

Affixing the suffix “-ene” to a group indicates the group is a divalent moiety, e.g., alkylene is an optionally substituted divalent moiety of alkyl (e.g., unsubstituted C₆ alkylene is represented by —(CH₂)₆—) and heteroalkylene is the divalent moiety of heteroalkyl (e.g., unsubstituted hetero-C₆-alkylene is represented by, for example, —(CH₂)—O—(CH₂)₅—).

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —SH, —SR^{aa}, —C(═O)H, —C(═O)R^{aa}, —CO₂H, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —C(═O)SR^{aa}, —SC(═O)SR^{aa}, —OC(═O)SR^{aa}, —SC(═O)OR^{aa}, —SC(═O)R^{aa}, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl;
or two geminal hydrogens on a carbon atom are replaced with the group ═O or ═S, wherein:
   each instance of R^{aa} is, independently, selected from C₁₋₆ alkyl, C₁₋₆ perhaloalkyl, C₁₋₆ alkenyl, C₁₋₆ alkynyl, heteroC₁₋₆ alkyl, heteroC₁₋₆alkenyl, heteroC₁₋₆alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —C(═O)R^{aa}, —C(═O)N(R^{cc})₂, —CO₂R^{aa}, —SO₂R^{aa}, —SO₂N(R^{cc})₂, —SO₂R^{cc}, —SO₂OR^{cc}, —SOR^{aa}, —C(═O)SR^{cc}, C₁₋₆ alkyl, C₁₋₆ perhaloalkyl, C₁₋₆ alkenyl, C₁₋₆ alkynyl, heteroC₁₋₆alkyl, heteroC₁₋₆alkenyl, heteroC₁₋₆alkynyl, C₃₋₁₀ carbocyclyl, 3-14 membered heterocyclyl, C₆₋₁₄ aryl, and 5-14 membered heteroaryl;
   each instance of R^{cc} is, independently, selected from hydrogen, C₁₋₆ alkyl, C₁₋₆ perhaloalkyl, C₁₋₆ alkenyl, C₁₋₆ alkynyl, heteroC₁₋₆ alkyl, heteroC₁₋₆ alkenyl, heteroC₁₋₆ alkynyl, C₃₋₁₀ carbocyclyl, 3-14 membered heterocyclyl, C₆₋₁₄ aryl, and 5-14 membered heteroaryl; and
   each X⁻ is a counterion.

In certain embodiments, each substituent is independently halogen, substituted or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each substituent is independently halogen, substituted or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each substituent is independently halogen, substituted or unsubstituted C₁₋₆ alkyl, —OR^{aa}, —N(R^{bb})₂, —CN, or —NO₂.

In certain embodiments, each substituent is independently halogen, substituted or unsubstituted C₁₋₁₀ alkyl, —OR^{aa}, —SR^{aa}, —N(R^{bb})₂, —CN, —SCN, or —NO₂, wherein R^{aa} is hydrogen, substituted or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, each nitrogen atom substituent is independently substituted or unsubstituted C₁₋₆ alkyl, —C(═O)R^{aa}, —CO₂R^{aa}, —C(═O)N(R^{bb})₂, or a nitrogen protecting group.

Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group.

Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, at least one oxygen protecting group is silyl, TBDPS, TBDMS, TIPS, TES, TMS, MOM, THP, t-Bu, Bn, allyl, acetyl, pivaloyl, or benzoyl.

The term “silyl” refers to the group —Si(R^{aa})₃, wherein R^{aa} is as defined herein.

In certain embodiments, each sulfur atom substituent is independently substituted or unsubstituted C₁₋₁₀ alkyl, —C(═O)R^{aa}, —CO₂R^{aa}, —C(═O)N(R^{bb})₂, or a sulfur protecting group.

In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severeity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for binding sigma receptors and/or dopamine transporters. In certain embodiments, a therapeutically effective amount is an amount sufficient for treating stimulant (e.g., psychostimulant) abuse, addiction, or dependence, or other indications and diseases associated with sigma receptors and/or dopamine transporters.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein the term “inhibit” or “inhibition” in the context of protein(s), for example, in the context of sigma receptors and/or dopamine transporter, refers to a reduction in the activity of the protein. In some embodiments, the term refers to a reduction of the level of protein(s) activity, e.g., (sigma receptors and/or dopamine transporter) activity, to a level that is statistically significantly lower than an initial level, which may, for example, be a baseline level of protein activity. In some embodiments, the term refers to a reduction of the level of protein activity, e.g., sigma receptors and/or dopamine transporter activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of protein activity.

As used herein, “dopamine transporter”, “dopamine active transporter”, and “DAT” refer to membrane-spanning protein that pumps dopamine from the synaptic cleft into cytosol. Reuptake of dopamine through DAT provides a primary mechanism for dopamine clearance from synapses. DAT is involved in a number of disorders including, but not limited to substance use/abuse disorders (i.e., addiction (e.g., alcoholism, cocaine addiction)), mood disorders (e.g., depression, bipolar disorder), and mental health disorders (e.g., attention-deficit/hyperactivity disorder, eating disorders).

“Dopamine” is the common name for 3,4-dihydroxyphenethylamine, a small molecule neurotransmitter. There are many dopamine pathways, including one that is associated with the motivational aspect of reward-motivated behavior (i.e., anticipation of a reward increases the level of dopamine in the brain). Dopamine exerts its function through direct binding to and activating dopamine receptors, five subtypes of dopamine receptors that have been identified (D1, D2, D3, D4, and D5), and all of these belong to the G-protein coupled receptor superfamily. Various nervous system diseases and disorders are associated the various dopamine systems.

The term “sigma receptor” or “σ-receptors” are protein cell surface receptors. There are two subtypes of sigma receptors: sigma-1 (σ₁) and sigma-2 (σ₂). Sigma receptors are a well-defined unique class of receptors, distinct from opioid receptors and phencyclidine binding sites (though originally sigma receptors were in fact thought to be opioid receptors). Various moieties are known to bind to sigma receptors including, for example, cocaine, methamphetamine, morphine, diacetylmorphine, opipramol, PCP, fluvoxamine, berberine, and dextromethorphan.

The terms “substance abuse disorders” and “substance use disorder” are used interchangeably to refer to a mental disorder affecting a person's brain and behavior, resulting in the inability to control their use of substances, including psychoactive substances either licit or illicit (e.g., cocaine, methamphetamine, ecstasy, alcohol, opioids, stimulants, psychedelics). Severe substance use disorders may be referred to as addictions.

The terms “substance addiction” refers to severe forms of substance use disorders. Drug addiction is a progressive disease resulting in losing control of the use of one or more substances (e.g., drugs, pharmaceuticals/medications, stimulants) despite consequences of that use. A disease of the mind characterized by compulsive engagement in rewarding or addictive stimuli. An addiction often involves addictive stimuli that are reinforcing (e.g., increase the likelihood that a person will seek repeated exposure to the agent causing the stimulus) and intrinsically rewarding (e.g., they are perceived by a person as being inherently desirable, positive, and pleasurable). The addiction may arise through transcriptional or epigenetic mechanisms and generally develops over time as a result of persistent exposure to addictive stimulus or stimuli. Cognitive control, particularly inhibitory control over behavior, is impaired in a person suffering from addiction. Additionally, stimulus-driven behavioral responses (i.e., stimulus control) that are associated with a particular rewarding stimulus tend to dominate the behavior of a person suffering from addiction. The term addiction encompasses addiction to drugs (e.g., cocaine, opioids, and the like), alcohol, gambling, etc. In certain embodiments, the addiction is a drug addiction. In certain embodiments, the addiction is a cocaine addiction. In certain embodiments, the addiction is a methamphetamine addiction. In certain embodiments the addiction is an ecstasy addiction. In certain embodiments, the addiction is an ethanol addiction. In certain embodiments, the addiction is an opioid addiction.

The terms “substance intake” refers to the consumption of substances (e.g., drugs, pharmaceuticals/medications, stimulants).

The term “substance” as used herein refers to psychoactive compounds or molecules. Psychoactive compounds and molecules change nervous system function (i.e., brain function) and result in alterations in mood, awareness, thoughts, feelings, and/or behavior. As used herein, common substances include drugs, pharmaceuticals/medications, and other stimulants, including but not limited to, alcohol, caffeine, nicotine, marijuana, certain pain medicines, heroin, LSD, cocaine, and amphetamines (e.g., methamphetamine, 3,4-methylenedioxymethamphetamine, cathinone). In some embodiments, the substance is methamphetamine or cocaine.

The term “stimulant” refers to compounds/molecules that increase activity of the central nervous system and/or body, are pleasurable and invigorating, and/or have sympathomimetic effects. A non-exhaustive list of exemplary stimulants include methylphenidate, amphetamines (e.g., methamphetamine), and cocaine. In some embodiments, the stimulant is methamphetamine or cocaine.

These and other exemplary substituents are described in more detail in the Detailed Description, Examples, and Claims. The invention is not limited in any manner by the above exemplary listing of substituents or definitions. Additional terms may be defined in other sections of this disclosure.

### DETAILED DESCRIPTION

Provided herein are compounds, compositions, methods, uses, and kits comprising compounds of Formula (I).

### Compounds

Provided herein are compounds of Formula (I):

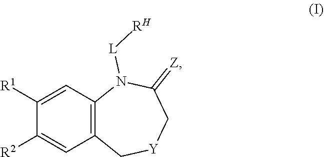

or pharmaceutically acceptable salt thereof, wherein:
   Z is O, S, NR^{N}, or C(R^{C})₂;
   each R^{N} is independently hydrogen, C₁₋₆ alkyl, or a nitrogen protecting group;
   each R^{C} is independently hydrogen or C₁₋₆ alkyl;
   Y is O, S, NR^{N}, or C(R^{C})₂;
   R¹ hydrogen, halogen, substituted or unsubstituted C₁₋₁₂ alkyl;
   R² hydrogen, halogen, substituted or unsubstituted C₁₋₁₂ alkyl;
   L is substituted or unsubstituted C₁₋₁₂ alkylene or substituted or unsubstituted hetero-C₁₋₁₂-alkylene;
   R^{H} is selected from the group consisting of:

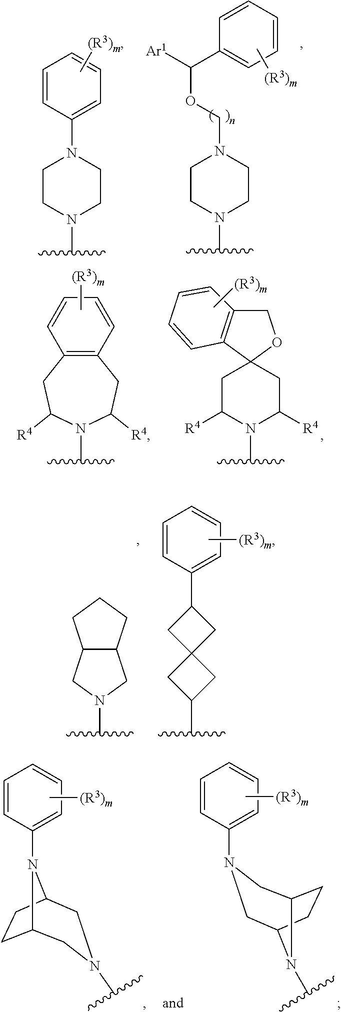

Ar¹ is substituted or unsubstituted aryl, or substituted or unsubstituted heteroaryl;
n is an integer selected from 1, 2, 3, 4, and 5;
each R³ is independently selected from the group consisting of hydrogen, halogen, —NO₂, —CN, —C(═O)R^{C}, and —C(═O)OR^{C};
each R⁴ is independently hydrogen or C₁₋₆ alkyl; and
m is an integer selected from 0, 1, 2, 3, 4, and 5.

In some embodiments, a compound of Formula (I) is a compound of Formula (IA) or (IB):

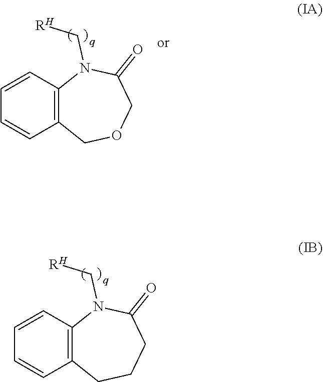

wherein q is an integer selected from 1, 2, 3, 4, 5, or 6.

In some embodiments, a compound of Formula (I) is a compound of Formula (IA), (IB), or (IC):

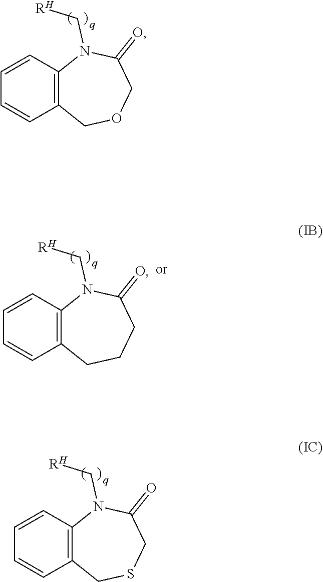

wherein q is an integer selected from 1, 2, 3, 4, 5, or 6. In some embodiments, q is 4.

In certain embodiments, a compound of Formula (I) is a compound of Formula (IA-1), (IA-2), (IB-1), or (IB-2):

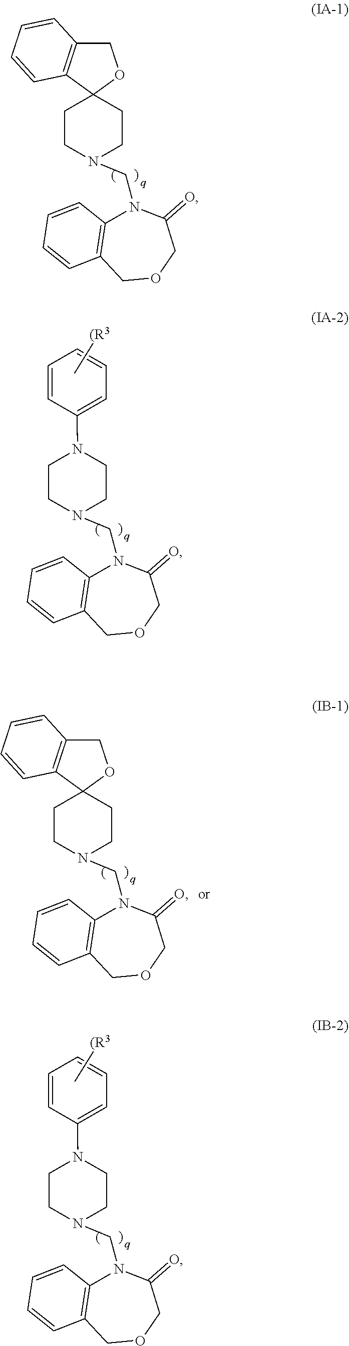

wherein q is an integer selected from 1, 2, 3, 4, 5, or 6.

In certain embodiments, a compound of Formula (I) is a compound of Formula (IA-1), (IA-2), (IB-1), (IB-2), (IC-1), or (IC-2):

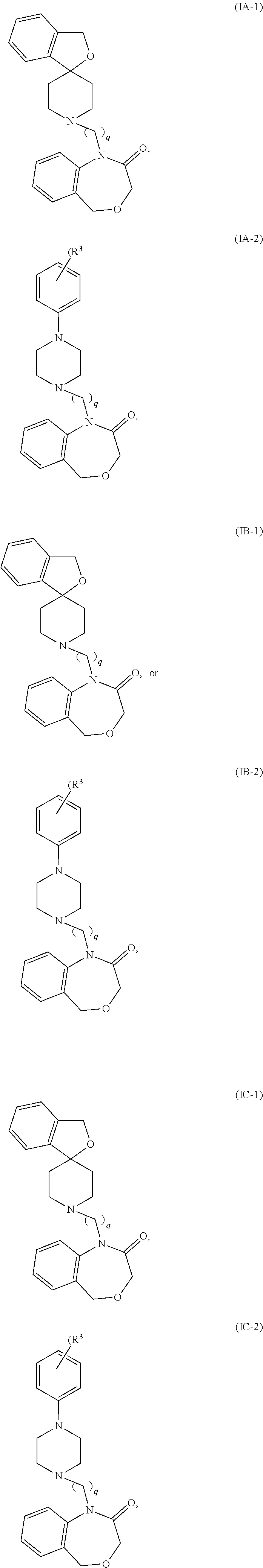

wherein q is an integer selected from 1, 2, 3, 4, 5, or 6. In some embodiments, q is 4.

In some embodiments, Z is O, S, NR^{N}, or C(R^{C})₂. In some embodiments Z is O, S, NH, or CH₂. In some embodiments, Z is O or S. In some embodiments, Z is O.

In some embodiments, Y is O, S, NR^{N}, or C(R^{C})₂. In some embodiments, Y is O, S, NH, NCH₃, CH₂, C(CH₃)H, or C(CH₃)₂. In some embodiments Y is O or CH₂. In certain embodiments, Y is O. In some embodiments, Y is CH₂.

In some embodiments, R¹ is hydrogen, halogen, substituted or unsubstituted C₁₋₁₂ alkyl. In some embodiments, R¹ is hydrogen, bromo, fluoro, or substituted or unsubstituted C₁₋₈ alkyl. In some embodiments, R¹ is hydrogen, bromo, fluoro, or substituted or unsubstituted C₁₋₆ alkyl. In some embodiments, R¹ is hydrogen, bromo, fluoro, or unsubstituted C₁₋₆ alkyl. In some embodiments, R¹ is hydrogen. In some embodiments, R¹ is bromo. In some embodiments, R¹ is fluoro. In some embodiments, R¹ is substituted or unsubstituted C₁₋₈ alkyl. In some embodiments, R¹ is substituted C₁₋₈ alkyl. In some embodiments, R¹ is C₁₋₈ haloalkyl. In some embodiments, R¹ is —CF₃. In some embodiments, R¹ is unsubstituted C₁₋₈ alkyl. In certain embodiments, R¹ is hydrogen.

In some embodiments, R² is hydrogen, halogen, substituted or unsubstituted C₁₋₁₂ alkyl. In some embodiments, R² is hydrogen, bromo, fluoro, or substituted or unsubstituted C₁₋₈ alkyl. In some embodiments, R² is hydrogen, bromo, fluoro, or substituted or unsubstituted C₁₋₆ alkyl. In some embodiments, R² is hydrogen, bromo, fluoro, or unsubstituted C₁₋₆ alkyl. In some embodiments, R² is hydrogen. In some embodiments, R² is bromo. In some embodiments, R¹ is fluoro. In some embodiments, R² is substituted or unsubstituted C₁₋₈ alkyl. In some embodiments, R² is substituted C₁₋₈ alkyl. In some embodiments, R² is C₁₋₈ haloalkyl. In some embodiments, R² is —CF₃. In some embodiments, R² is unsubstituted C₁₋₈ alkyl. In certain embodiments, R² is hydrogen.

In some embodiments, R^{H} is selected from the group consisting of:

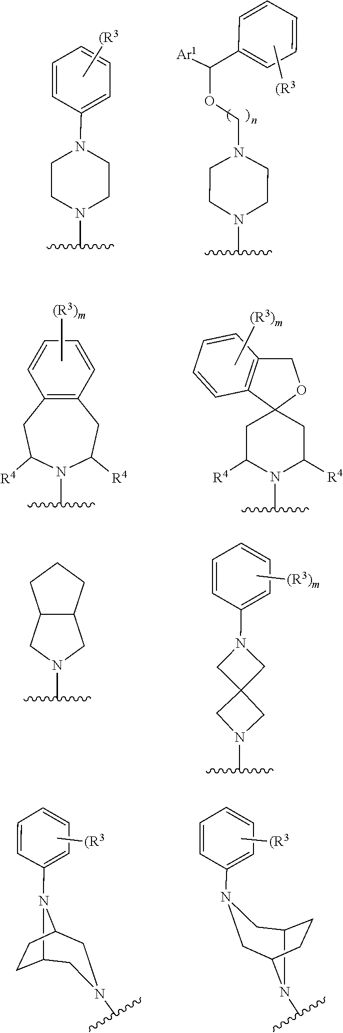

In some embodiments, R^{H} is selected from the group consisting of:

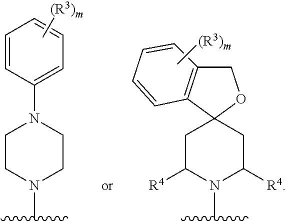

In certain embodiments, R^{H} is:

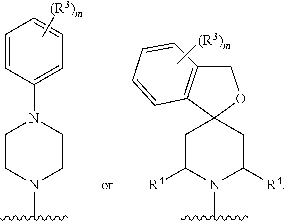

In some embodiments, R^{H} is:

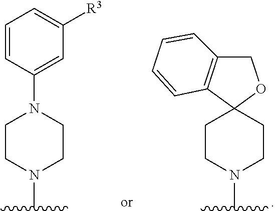

In some embodiments, Ar¹ is substituted or unsubstituted aryl, or substituted or unsubstituted heteroaryl. In certain embodiments, Ar¹ is substituted or unsubstituted aryl. In certain embodiments, Ar¹ is substituted or unsubstituted heteroaryl. In certain embodiments, Ar¹ is substituted or unsubstituted 5- or 6-membered heteroaryl. In certain embodiments, Ar¹ is phenyl, thiopheneyl, thienyl, furanyl, pyrrolyl, imidazolyl, pyrazolyl, oxazolyl, isoxazolyl, thiazolyl, or isothiazolyl. In some embodiments, Ar¹ is phenyl or thiopheneyl or thienyl. In some embodiments, Ar¹ is phenyl. In some embodiments, Ar¹ is thiopheneyl or thienyl. In certain embodiments, Ar¹ is

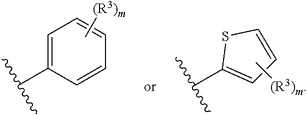

In certain embodiments, Ar¹ is

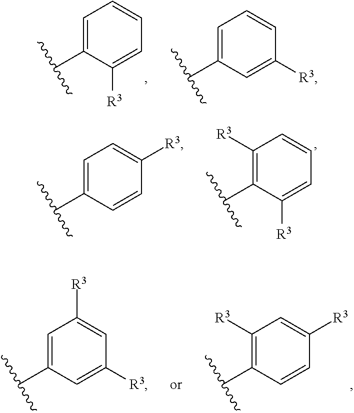

In some embodiments, R³ is independently selected from the group consisting of hydrogen, halogen, —NO₂, —CN, —C(═O)R^{C}, and —C(═O)OR^{C}. In certain embodiments, R³ is hydrogen, halogen, —NO₂, —CN, —C(═O)H, —C(═O) CH₃, —C(═O)OH, and —C(═O)OR^{C}. In certain embodiments, R³ is hydrogen or halogen. In certain embodiments, R³ is halogen. In certain embodiments, R³ is hydrogen. In certain embodiments, R³ is —NO₂. In certain embodiments, R³ is —CN. In certain embodiments, R³ is —C(═O)H. In certain embodiments, R³ is —C(═O) CH₃. In certain embodiments, R³ is —C(═O)OH. In certain embodiments, R³ is —C(═O)OR^{C}.

In some embodiments, m is an integer selected from 0, 1, 2, 3, 4, and 5. In some embodiments, m is 0, 1, 2, or 3. In some embodiments, m is 2. In certain embodiments, m is 3. In certain embodiments, m is 4. In some embodiments, m is 5. In some embodiments, m is 0 or 1. In some embodiments, m is 1. In certain embodiments, m is 0.

In some embodiments, n is an integer selected from 1, 2, 3, 4, and 5. In some embodiments, n is 1. In some embodiments, n is 2. In certain embodiments, n is 3. In certain embodiments, n is 4. In some embodiments, n is 5.

In some embodiments, each R⁴ is independently hydrogen or C₁₋₆ alkyl. In some embodiments, R⁴ is hydrogen. In some embodiments, R⁴ is C₁₋₆ alkyl. In some embodiments, R⁴ is methyl, ethyl, n-propyl, isopropyl, n-butyl, sec-butyl, isobutyl, or n-pentyl. In some embodiments, all instances of R⁴ are hydrogen. In some embodiments, all instances of R⁴ are methyl. In some embodiments, some instances of R⁴ are hydrogen and some are methyl.

In certain embodiments, L is substituted or unsubstituted C₁₋₁₂ alkylene or substituted or unsubstituted hetero-C₁₋₁₂-alkylene. In some embodiments, L is substituted or unsubstituted C₁₋₁₂ alkylene. In some embodiments, L is substituted or unsubstituted hetero-C₁₋₁₂-alkylene. In some embodiments, L is substituted or unsubstituted C₁₋₆ alkylene. In some embodiments, L is substituted or unsubstituted hetero-C₁₋₆-alkylene. In some embodiments, L is unsubstituted C₁₋₆ alkylene. In some embodiments, L is unsubstituted hetero-C₁₋₆-alkylene. In some embodiments, L is substituted C₁₋₆ alkylene. In some embodiments, L is substituted hetero-C₁₋₆-alkylene. In certain embodiments, L is C₁₋₆ alkylene. In some embodiments, L is C₄ alkylene. In some embodiments, L is hetero-C₁₋₆-alkylene. In some embodiments, L is hetero-C₁₋₆-alkylene comprising at least 1 oxygen atom. In some embodiments, L is hetero-C₁₋₆-alkylene comprising one oxygen atom. In some embodiments, L is hetero-C₁₋₆-alkylene comprising two oxygen atoms. In some embodiments, L is

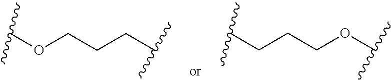 [SMILES: *CCCO* ; *CCCO*]

In some embodiments, q is an integer selected from 1, 2, 3, 4, 5, or 6. In some embodiments, m is 1. In some embodiments, m is 2. In certain embodiments, m is 3. In certain embodiments, m is 4. In some embodiments, m is 5. In some embodiments, m is 6.

In some embodiments, each R^{C} is independently hydrogen or C₁₋₆ alkyl. In some embodiments, R^{C} is hydrogen. In some embodiments, R^{C} is C₁₋₆ alkyl. In some embodiments, R^{C} is methyl, ethyl, n-propyl, isopropyl, n-butyl, sec-butyl, isobutyl, or n-pentyl. In some embodiments, all instances of R^{C} are hydrogen. In some embodiments, all instances of R^{C} are methyl. In some embodiments, some instances of R^{C} are hydrogen and some are methyl.

In some embodiments, each R^{N} is independently hydrogen, C₁₋₆ alkyl, or a nitrogen protecting group. In some embodiments, R^{N} is hydrogen. In some embodiments, R^{N} is C₁₋₆ alkyl. In some embodiments, R^{N} is methyl, ethyl, n-propyl, isopropyl, n-butyl, sec-butyl, isobutyl, or n-pentyl. In some embodiments, all instances of R^{N} are hydrogen. In some embodiments, all instances of R^{N} are methyl. In some embodiments, some instances of R^{N} are hydrogen and some are methyl. In certain embodiments, R^{N} is a nitrogen protecting group. In certain embodiments, R^{N} is a nitrogen protecting group selected from Fmoc, Boc, or Cbz.

In certain embodiments, a compound of Formula (I) is of the formula:

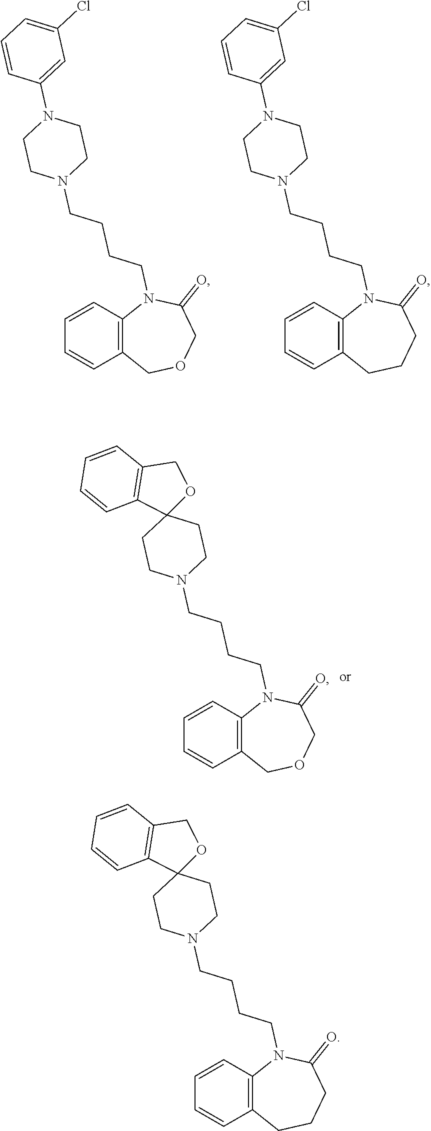 [SMILES: O=C1COCc2ccccc2N1CCCCN1CCN(c2cccc(Cl)c2)CC1 ; O=C1CCCc2ccccc2N1CCCCN1CCN(c2cccc(Cl)c2)CC1 ; O=C1COCc2ccccc2N1CCCCN1CCC2(CC1)OCc1ccccc12 ; O=C1CCCc2ccccc2N1CCCCN1CCC2(CC1)OCc1ccccc12]

In some embodiments, the compound is of the formula:

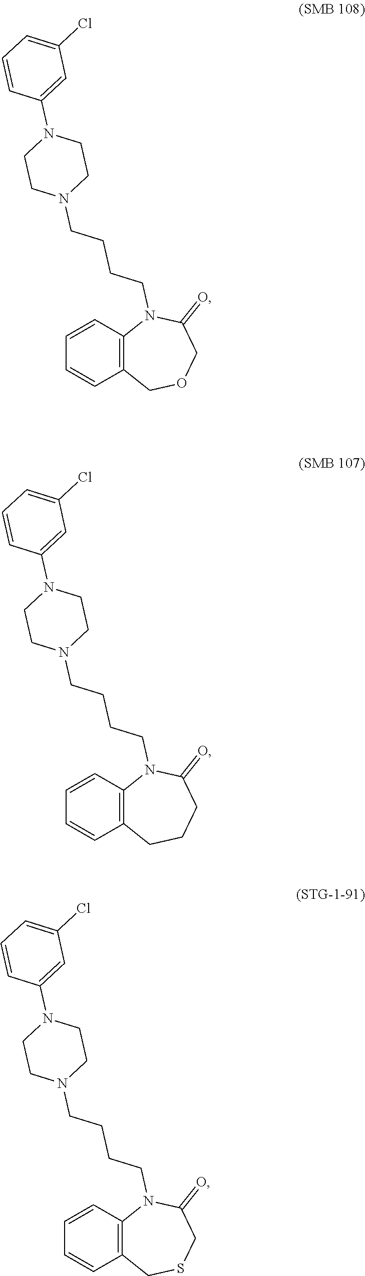 [SMILES: O=C1COCc2ccccc2N1CCCCN1CCN(c2cccc(Cl)c2)CC1 ; O=C1CCCc2ccccc2N1CCCCN1CCN(c2cccc(Cl)c2)CC1 ; O=C1CSCc2ccccc2N1CCCCN1CCN(c2cccc(Cl)c2)CC1]

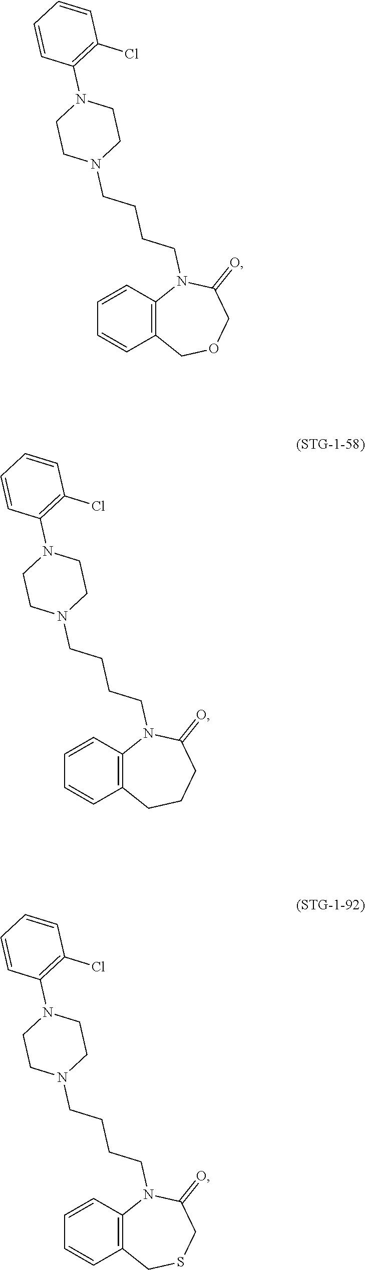 [SMILES: O=C1COCc2ccccc2N1CCCCN1CCN(c2ccccc2Cl)CC1 ; O=C1CCCc2ccccc2N1CCCCN1CCN(c2ccccc2Cl)CC1 ; O=C1CSCc2ccccc2N1CCCCN1CCN(c2ccccc2Cl)CC1]

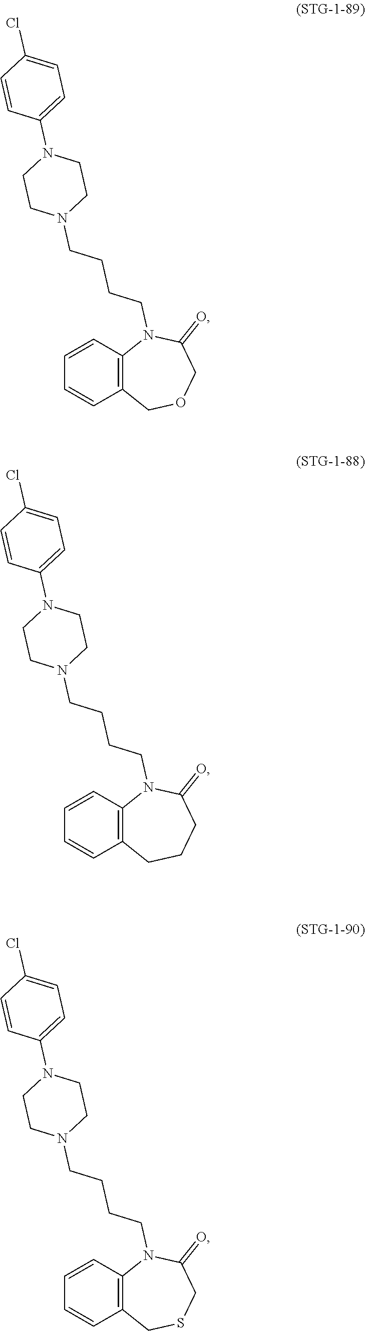 [SMILES: O=C1COCc2ccccc2N1CCCCN1CCN(c2ccc(Cl)cc2)CC1 ; O=C1CCCc2ccccc2N1CCCCN1CCN(c2ccc(Cl)cc2)CC1 ; O=C1CSCc2ccccc2N1CCCCN1CCN(c2ccc(Cl)cc2)CC1]

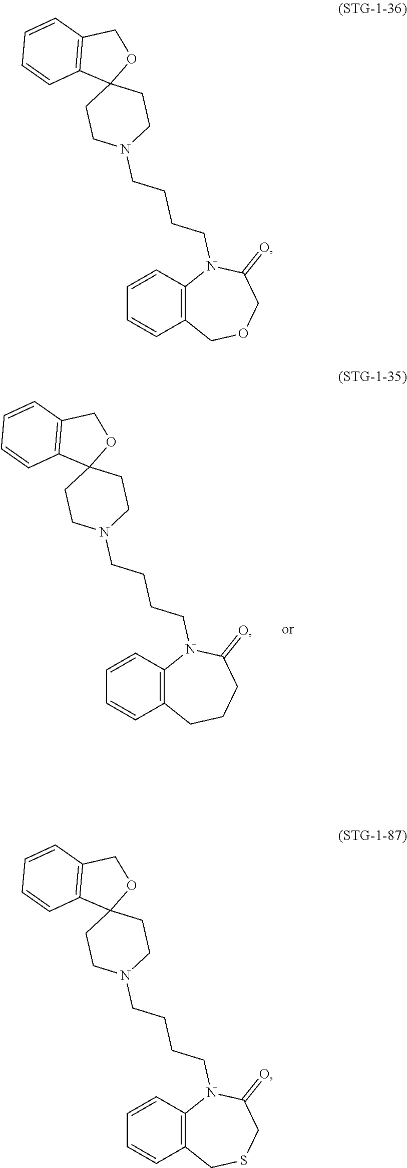 [SMILES: O=C1COCc2ccccc2N1CCCCN1CCC2(CC1)OCc1ccccc12 ; O=C1CCCc2ccccc2N1CCCCN1CCC2(CC1)OCc1ccccc12 ; "or" ; O=C1CSCc2ccccc2N1CCCCN1CCC2(CC1)OCc1ccccc12]

or pharmaceutically acceptable salt thereof.

In some embodiments, the compound is sigma-receptor antagonist. In certain embodiments, the compound is dopamine transporter inhibitor. In some embodiments, the compounds is a sigma-receptor antagonist and a dopamine transporter inhibitor.

### Compositions, Administration, and Kits

The present disclosure provides pharmaceutical compositions comprising a compound of Formula (I), or a pharmaceutically acceptable salt thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., proliferative disease, hematological disease, neurological disease, painful condition, psychiatric disorder, or metabolic disorder) in a subject in need thereof. In certain embodiments, the effective amount is an amount effective for inhibiting the activity (e.g., aberrant activity, such as increased activity) of a protein kinase in a subject or cell.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

In certain embodiments, the effective amount is an amount effective for inhibiting the activity of a protein by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of a sigma receptor and/or dopamine transporter by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of a sigma receptor and/or dopamine transporter by a range between a percentage described in this paragraph and another percentage described in this paragraph, inclusive.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone)(crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymers and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional components other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymers and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNA, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

The present disclosure also provides a kit comprising: a compound provided herein (e.g., a compound of Formula (I)), or pharmaceutically acceptable salt thereof, or a composition provided herein (e.g., a composition comprising a compound of Formula (I)); and instructions for administering the compound, or pharmaceutically acceptable salt thereof, or composition to a subject.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a protein kinase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

### Methods

Provided herein are methods utilizing and uses of the compounds disclosed herein (e.g., compounds of Formula (I)).

In an additional aspect, the present disclosure provides methods of treating or preventing substance intake by a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)). In some embodiments, the substance is cocaine or methamphetamine. In certain embodiments, the substance is cocaine. In certain embodiments, the substance is methamphetamine.

In another aspect, provided herein are methods of treating or preventing substance use disorder in a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)). In some embodiments, the substance is cocaine or methamphetamine. In certain embodiments, the substance is cocaine. In certain embodiments, the substance is methamphetamine.

In one aspect, the present disclosure provides methods of treating the symptoms of substance use disorder in a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)). In some embodiments, the substance is cocaine or methamphetamine. In certain embodiments, the substance is cocaine. In certain embodiments, the substance is methamphetamine.

In an additional aspect, the present disclosure provides methods of treating or preventing substance addiction in a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)). In some embodiments, the substance is cocaine or methamphetamine. In certain embodiments, the substance is cocaine. In certain embodiments, the substance is methamphetamine.

In another aspects, provided herein are methods of treating the symptoms of substance addiction in a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)). In some embodiments, the substance is cocaine or methamphetamine. In certain embodiments, the substance is cocaine. In certain embodiments, the substance is methamphetamine.

In one aspect, the present disclosure provides methods of treating or preventing neurotoxic effects resulting from substance use disorder, substance addiction, and/or substance intake by a subject comprising administering to the subject a therapeutically effective amount of a compound provided herein (e.g., a compound of Formula (I)), or pharmaceutically acceptable salt thereof, or a composition provided herein (e.g., a composition comprising a compound of Formula (I)). In some embodiments, the substance is cocaine or methamphetamine. In certain embodiments, the substance is cocaine. In certain embodiments, the substance is methamphetamine.

In a further aspect, provided herein are methods of treating or preventing a disease or disorder associated with one or more sigma receptors comprising administering to a subject a therapeutically effective amount of a compound provided herein (e.g., a compound of Formula (I)), or pharmaceutically acceptable salt thereof, or a composition provided herein (e.g., a composition comprising a compound of Formula (I)).

In an additional aspect, the present disclosure provides methods of treating or preventing a disease or disorder associated with one or more dopamine transporters comprising administering to a subject a therapeutically effective amount of a compound provided herein (e.g., a compound of Formula (I)), or pharmaceutically acceptable salt thereof, or a composition provided herein (e.g., a composition comprising a compound of Formula (I)).

In another aspects, provided herein are methods of treating or preventing a disease or disorder associated with one or more dopamine transporters and with one or more sigma receptors comprising administering to a subject a therapeutically effective amount of a compound provided herein (e.g., a compound of Formula (I)), or pharmaceutically acceptable salt thereof, or a composition provided herein (e.g., a composition comprising a compound of Formula (I)).

Another object of the present disclosure is the use of a compound as described herein (e.g., a compound of Formula (I)), or pharmaceutically acceptable salt thereof, or a composition provided herein (e.g., a composition comprising a compound of Formula (I)) in the manufacture of a medicament for use in the treatment of a disorder or disease described herein. Another object of the present disclosure is the use of a compound as described herein (e.g., a compound of Formula (I)), or pharmaceutically acceptable salt thereof, or a composition provided herein (e.g., a composition comprising a compound of Formula (I)) for use in the treatment of a disorder or disease described herein. Another object of the present disclosure is the use of a compound as described herein (e.g., a compound of Formula (I)), or pharmaceutically acceptable salt thereof, or a composition provided herein (e.g., a composition comprising a compound of Formula (I)) in the manufacture of a veterinary composition for use in the treatment or prevention of a disorder or disease in veterinary applications.

### Examples

In order that the present disclosure may be more fully understood, the following examples are set forth. The synthetic and biological examples described in this application are offered to illustrate the compounds, pharmaceutical compositions, methods, and uses provided herein and are not to be construed in any way as limiting their scope.

Unless otherwise noted, all reagents (chemicals and solvents) were commercially available and purchased through Thermo Fisher Scientific or Millipore Sigma and used without further purification. All reactions were monitored by thin-layer chromatography (TLC) on 0.2 mm silica plates and visualized under UV light 254 nm. Column chromatography was performed using Teledyne ISCO Combiflash. ¹H NMR and ¹³C NMR spectra were obtained using AVANCE NEO 600 MHz NMR spectrometer with N2-Cryo-Platform for PRODIGY probe in CDCl₃ unless otherwise noted. Chemical shifts are reported in parts per million (ppm).

### Synthesis

### Scheme 1

The spirocyclic amine ‘head’ group (3) was synthesized according to Scheme 1.

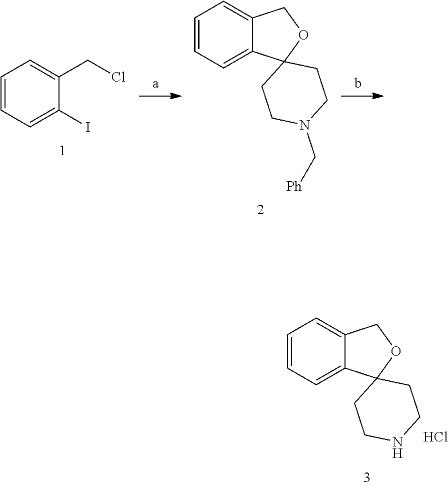 [SMILES: ClCc1ccccc1I ; c1ccc(CN2CCC3(CC2)OCc2ccccc23)cc1 ; c1ccc2c(c1)COC21CCNCC1 ; Cl]

Scheme 1. a) (i) iPrMgCl·LiCl, H₂, THF, −45° C., 1 hr (ii) 1-benzylpiperidin-4-one, −45° C. to RT, 3 hr; b) H₂, Pd/C 10%, EtOH, RT, 23 hr.

### Scheme 2

The newly synthesized N-substituted-1,5-dihydrobenzo[e][1,4]oxazepin-2(3H)-ones and 2,3,4,5-tetrahydro-1H-1-benzo[b]azepin-2-ones were prepared following Scheme 2.

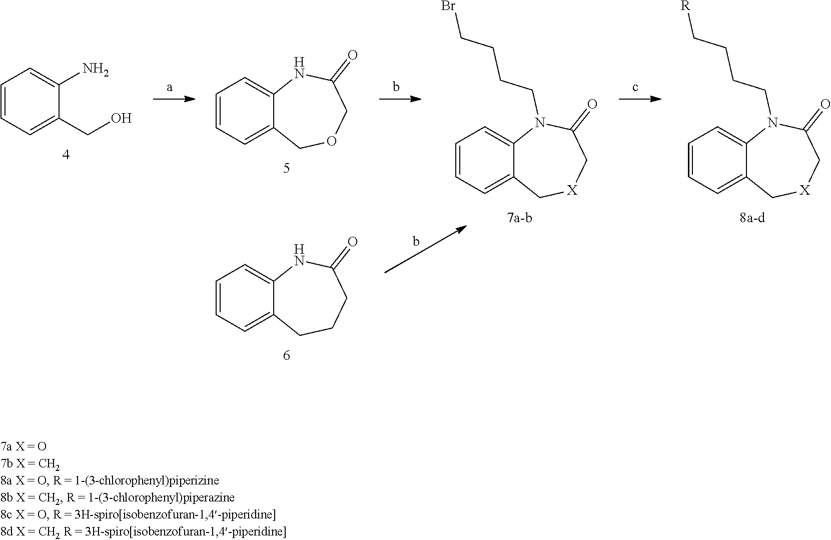

Scheme 2. a) (i) Chloroacetyl chloride, TEA, THE, 0° C. to RT, 2 hr. (ii) NaH, 2 hr; b) NaH, 1,4-dibromobutane, DMF, RT, 1 hr; c) select amines, K₂CO₃, DMF, 60° C. overnight.

### 1′-Benzyl-3H-spiro[isobenzofuran-1,4′-piperidine] (2)

1-(Chloromethyl)-2-iodobenzene (5.0 g, 19.8 mmol) was dissolved in anhydrous THF (80 mL) and cooled to −45° C. A 1.3 M solution of isopropylmagnesium chloride lithium chloride in THF (18.3 mL, 23.8 mmol) was added dropwise and the reaction was allowed to stir for 1 hour at −45° C. under argon gas. 1-Benzylpiperidin-4-one (3.5 mL, 19.8 mmol) was dissolved in THF (20 mL) and was added dropwise to the reaction mixture. The reaction mixture was then allowed to stir for 10 minutes at −45° C. and then was allowed to warm to room temperature. The reaction mixture continued to stir for 3 additional hours at room temperature. The reaction mixture was quenched with water (75 mL), extracted with ethyl acetate (75 mL), and the organic layer was washed with brine (3×100 mL). The organic layer was dried with sodium sulfate, filtered, and concentrated in vacuo to give the crude compound as a light brown oil. The crude compound was purified via column chromatography using a gradient of hexanes:ethyl acetate from 100:0 to 60:40 to give the desired compound 2 as a clear oil (4.0 g, 90%) which showed:

¹H NMR (600 MHZ, Chloroform-d) δ 7.39-7.15 (m, 9H), 5.07 (s, 2H), 3.60 (s, 2H), 2.85 (dd, J=10.0, 2.0 Hz, 2H), 2.44 (dt, J=12.0, 2.4 Hz, 2H), 2.01 (dt, J=13.2, 4.4 Hz, 2H), 1.76 (dd, J=14.4, 2.4 Hz, 2H).

¹³C NMR (151 MHZ, Chloroform-d) 145.78, 138.98 (2C), 129.43 (2C), 128.28 (2C), 127.59, 127.38, 127.10, 121.10, 120.89, 84.81, 70.78, 63.54, 50.21 (2C), 36.64 (2C).

### 3H-spiro[isobenzofuran-1,4′-piperidine] hydrochloride (3)

1′-Benzyl-3H-spiro[isobenzofuran-1,4′-piperidine] (4.0 g, 14.3 mmol) was dissolved in ethanol (100 mL) and Pd/C 10% (1.7 g, 15.7 mmol) was added. The reaction mixture was allowed to stir for 23 hours at room temperature under hydrogen gas. The reaction mixture was filtered using celite and concentrated. The resulting compound was then dissolved in methanol (100 mL) and a 4.0 M solution of HCl in 1,4-dioxane (35.8 mL) was added. The reaction mixture was allowed to stir at room temperature for 1 hour before being concentrated. Trituration was done using diethyl ether (15 mL) to give the desired product 3 as a white powder (2.8 g, 88%) which showed:

¹H NMR (600 MHZ, Methanol-d₄) δ 7.35-7.31 (m, 3H), 7.31-7.28 (m, 1H), 7.26-7.23 (m, 1H), 5.10 (s, 2H), 3.45-3.34 (m, 7H), 2.21 (dd, J=14.0, 5.0 Hz, 3H), 1.92 (dt, J=15.4, 4.7, 2.9 Hz, 3H).

¹³C NMR (151 MHz Methanol-d₄) δ 144.77, 140.04, 129.55, 128.83, 122.51, 83.39, 72.22, 42.32 (2C), 34.16 (2C).

### 1,5-Dihydrobenzo[e][1,4]oxazepin-2(3H)-one (5)

To a solution of 2-aminobenzyl alcohol (5.00 g, 0.041 mol) and triethylamine (5.7 mL, 0.041 mol) in anhydrous THF (20 mL) was added chloroacetyl chloride (3.25 mL, 0.040 mol) dropwise at 0° C. Reaction mixture was allowed to warm to room temperature and stirred while the N-acylated intermediate formed. After two hours, all starting material had been consumed as seen by TLC, and NaH from 60% dispersion in mineral oil (4.05 g, 0.102 mol) was added portion wise. Intramolecular cyclization was completed after stirring at RT for an additional 2 hours. Reaction mixture was quenched with methanol and concentrated under vacuum. The resulting residue was dissolved in EtOAc (100 mL) and washed with water (3×50 mL) and brine (1×20 mL). The organic layer was dried over Na₂SO₄ and concentrated under vacuum. The crude oil was purified by silica gel column (EtOAc in Hex from 50 to 80%) to afford 5 as an ivory powder (4.0 g, 60%).

¹H NMR (600 MHZ, CDCl₃) δ 9.07 (s, 1H), 7.32-7.25 (td, 1H), 7.12 (d, J=7.5, 1.7 Hz, 1H), 7.05 (td, J=7.5, 1.2 Hz, 1H), 7.01 (d, J=7.9, 1.1 Hz, 1H), 4.74 (s, 2H), 4.60 (s, 2H).

¹³C NMR (151 MHZ, CDCl₃) δ 173.87, 136.11, 129.34, 128.92, 128.60, 123.85, 119.57, 73.68, 73.01.

### 1-(4-Bromobutyl)-1,5-dihydrobenzo[e][1,4]oxazepin-2(3H)-one (7a)

To a solution of 5 (200 mg, 1.23 mmol) in anhydrous DMF (12 mL) was added NaH from a 60% dispersion in mineral oil (97.9 mg, 2.45 mmol) and 1,4-dibromobutane (0.16 mL, 1.35 mmol) and was left to stir at RT for 30 minutes. The reaction mixture was quenched with water (200 mL), extracted with EtOAc (3×50 mL), and the combined organic layers washed with brine (1×20 mL), and dried over Na₂SO₄ before concentrating under vacuum. The crude oil was purified by silica gel column (EtOAc in Hex from 50 to 70%) to afford compound 7a as a pale-yellow oil (320 mg, 87%).

¹H NMR (600 MHZ, CDCl₃) δ 7.48 (td, J=7.7, 1.6 Hz, 1H), 7.37 (dd, J=7.8, 1.3 Hz, 1H), 7.30 (d, J=7.7 Hz, 1H), 7.28 (dd, J=7.8, 0.9 Hz, 1H), 4.64 (s, 2H), 3.98 (t, J=7.4 Hz, 1H), 3.96 (s, 2H), 3.40 (t, J=6.6 Hz, 2H), 1.90-1.84 (m, 2H), 1.81-1.74 (m, 2H).

¹³C NMR (151 MHZ, CDCl₃) δ 168.09, 142.17, 130.69, 130.22, 129.52, 126.65, 120.99, 67.87, 67.53, 46.01, 33.48, 30.08, 26.72.

### 1-(4-Bromobutyl)-1,3,4,5-tetrahydro-2H-benzo[b]azepin-2-one (7b)

To a solution of commercially available 1,3,4,5-tetrahydro-2H-benzo[b]azepin-2-one 6 (500 mg, 3.10 mmol) in anhydrous DMF (24 mL) was added NaH from a 60% dispersion in mineral oil (248 mg, 6.20 mmol) and 1,4-dibromobutane (0.40 mL, 3.40 mmol) and was left to stir at RT for 30 minutes. The reaction mixture was quenched with water (300 mL), extracted with EtOAc (3×100 mL), and the combined organic layers washed with brine (1×20 mL), and dried over Na₂SO₄ before concentrating under vacuum. The crude oil was purified by silica gel column (EtOAc in Hex from 50 to 70%) to afford to compound 7b as a pale-yellow oil (620 mg, 67%).

¹H NMR (601 MHZ, CDCl₃) δ 7.29 (td, J=7.7, 1.8 Hz, 1H), 7.20 (td, J=7.0, 1.7 Hz, 1H), 7.18 (dd, J=6.2, 1.0 Hz, 1H), 7.16 (dd, J=7.3, 1.1 Hz, 1H), 4.39-3.55 (m, 2H), 3.37 (d, J=6.7 Hz, 2H), 2.71 (s, 2H), 2.26 (t, J=6.9 Hz, 2H), 2.16 (s, 2H), 1.88-1.80 (m, 2H), 1.74-1.66 (m, 2H).

¹³C NMR (151 MHZ, CDCl₃) δ 173.12, 142.31, 136.10, 129.57, 127.75, 126.46, 122.73, 46.83, 33.39, 33.25, 30.43, 30.28, 28.99, 27.11.

### 1-(4-(4-(3-Chlorophenyl) piperazin-1-yl)butyl)-1,5-dihydrobenzo[e][1,4]oxazepin-2(3H)-one (8a, SMB108)

To a solution of 7a (115 mg, 0.394 mmol) in anhydrous DMF (4.00 mL) was added 1-(3-chlorophenyl) piperazine hydrochloride (92.0 mg, 0.391 mmol) and K₂CO₃ (0.546 g, 3.94 mmol). The reaction mixture was left to stir overnight at 60° C. The reaction mixture was quenched with water (150 mL), extracted with EtOAc (3×50 mL), and the combined organic layers washed with brine (1×20 mL), and dried over Na₂SO₄ before concentrating under vacuum. The crude oil was purified by silica gel column (EtOAc in Hex from 20 to 100%) to afford compound 8a as a clear oil (120 mg, 74%).

¹H NMR (600 MHZ, CDCl₃) δ 7.46 (td, J=8.2, 1.5 Hz, 1H), 7.36 (dd, J=7.5, 1.6 Hz, 1H), 7.28 (ddd, J=7.3, 5.4, 1.1 Hz, 2H), 7.15 (t, J=8.1 Hz, 1H), 6.85 (t, J=2.2 Hz, 1H), 6.79 (ddd, J=8.0, 2.0, 0.8 Hz, 1H), 6.76 (ddd, J=8.5, 2.5, 0.8 Hz, 1H), 4.64 (s, 2H), 3.96 (s, 2H), 3.95 (t, J=7.7 Hz, 1H), 3.16 (dd, J=6.0, 3.6 Hz, 4H), 2.53 (dd, J=6.5, 4.1 Hz, 4H), 2.37 (dd, J=8.4, 6.5 Hz, 2H), 1.70-1.62 (m, 2H), 1.57-1.49 (m, 2H).

¹³C NMR (151 MHz, CDCl₃) δ 168.06, 152.32, 142.46, 134.57, 130.66, 130.14, 130.01, 129.59, 127.01, 121.07, 119.24, 116.51, 113.21, 67.91, 67.66, 57.85 (2C), 52.96 (2C), 48.61, 46.94, 26.01, 24.21.

### 1-(4-(4-(3-Chlorophenyl) piperazin-1-yl)butyl)-1,3,4,5-tetrahydro-2H-benzo[b]azepin-2-one (8b, SMB107)

To a solution of 7b (200 mg, 0.675 mmol) in anhydrous DMF (7.00 mL) was added 1-(3-chlorophenyl) piperazine hydrochloride (157 mg, 0.68 mmol) and K₂CO₃ (0.930 g, 6.8 mmol). The reaction mixture was left to stir overnight at 60° C. The reaction was quenched with water (150 mL), extracted with EtOAc (3×50 mL), and the combined organic layers washed with brine (1×20 mL), and dried over Na₂SO₄ before concentrating under vacuum. The crude oil was purified by silica gel column (EtOAc in Hex from 20 to 100%) to afford compound 8b as a clear oil (202 mg, 73%).

¹H NMR (600 MHZ, CDCl₃) δ 7.29 (dd, J=7.8, 1.0 Hz, 1H), 7.23-7.18 (m, 2H), 7.18-7.11 (m, 2H), 6.85 (t, J=2.2 Hz, 1H), 6.77 (ddd, J=10.7, 8.5, 2.3 Hz, 2H), 3.94-3.47 (m, 2H), 3.16 (dd, J=6.7, 3.7 Hz, 4H), 2.71 (s, 2H), 2.53 (dd, J=5.9, 3.7 Hz, 4H), 2.36 (dd, J=8.8, 6.7 Hz, 2H), 2.26 (d, J=6.8 Hz, 2H), 2.22-2.08 (m, 2H), 1.64-1.55 (m, 2H), 1.53-1.46 (m, 2H).

¹³C NMR (151 MHZ, CDCl₃) δ 173.02, 152.46, 142.50, 136.12, 135.04, 130.11, 129.47, 127.66, 126.34, 122.80, 119.29, 115.79, 113.92, 58.15 (2C), 53.05 (2C), 48.69, 47.66, 33.41, 30.25, 29.02, 26.31, 24.38.

### 1-(4-(3H-spiro[isobenzofuran-1,4′-piperidin]-1′-yl)butyl)-1,3,4,5-tetrahydro-2H-benzo[b]azepin-2-one (8c, STG-1-36 (previously STG-1-26))

1-(4-Bromobutyl)-1,3,4,5-tetrahydro-2H-benzo[b]azepin-2-one (156 mg, 0.5 mmol) and 3 (113 mg, 0.5 mmol) were dissolved in DMF (5 mL) and potassium carbonate (173 mg, 2.5 mmol) was added. The reaction mixture was heated to 60° C. and was allowed to stir for 5 hours before more potassium carbonate was added (518 mg, 7.5 mmol). The reaction mixture was allowed to stir for another 31 hours. The reaction mixture was quenched with water (10 mL), extracted with ethyl acetate (20 mL), and the organic layer was washed with brine (3×20 mL). The organic layer was collected, dried with sodium sulfate, filtered, and concentrated in vacuo to give the crude compound as a light orange oil. The crude compound was purified via column chromatography using a gradient of hexane:ethyl acetate 100:0 to 0:100 to give the desired compound 8c as a clear oil (66 mg, 31%) which showed:

¹H NMR (600 MHZ, Chloroform-d) δ 7.47 (ddd, J=8.1, 7.4, 1.6 Hz, 1H), 7.36 (dd, J=7.6, 1.6 Hz, 1H), 7.31-7.24 (m, 4H), 7.21-7.18 (m, 1H), 7.14-7.10 (m, 1H), 5.06 (s, 2H), 4.64 (s, 2H), 3.97 (s, 2H), 3.96 (d, J=7.6 Hz, 2H), 2.84-2.77 (m, 2H), 2.40 (t, J=7.6 Hz, 2H), 2.35 (dt, J=12.0, 2.2 Hz, 2H), 1.95 (td, J=13.3, 4.6 Hz, 2H), 1.76 (ddd, J=14.0, 5.1, 2.4 Hz, 2H), 1.68-1.62 (m, 2H), 1.58-1.52 (m, 2H).

¹³C NMR (151 MHZ, CDCl₃) δ 168.16, 145.81, 142.65, 139.07, 130.78, 130.27, 129.75, 127.71, 127.49, 126.65, 121.24, 121.20, 120.95, 84.84, 70.88, 68.06, 67.81, 58.45, 50.28 (2C), 47.22, 36.75 (2C), 26.35, 24.64.

### 1-(4-(3H-spiro[isobenzofuran-1,4′-piperidin]-1′-yl)butyl)-1,5-dihydrobenzo[e][1,4]oxazepin-2(3H)-one (8d, STG-1-35 (previously STG-1-28))

1-(4-Bromobutyl)-1,5-dihydrobenzo[e][1,4]oxazepin-2(3H)-one (260 mg, 0.88 mmol) and 3 (198 mg, 0.88 mmol) were dissolved in DMF (8 mL) and potassium carbonate (1.2 g, 8.8 mmol) was added. The reaction mixture was heated to 60° C. and was allowed to stir for 36 hours. The reaction mixture was quenched with water (10 mL), extracted with ethyl acetate (20 mL), and the organic layer was washed with brine (3×20 mL). The organic layer was collected, dried with sodium sulfate, filtered, and concentrated in vacuo to give the crude compound as a light orange oil.

### Biological Data

12 compounds in binding assays (either sigma 1 or sigma 2 receptors) at 1.0×10⁻⁶ M. Compound binding was calculated as a % inhibition of the binding of a ligand specific for each target.

TABLE A

| | | | | | | | Non | | Detection | |
|---|---|---|---|---|---|---|---|---|---|---|
| Assay | Source | Ligand | Conc. | | Kd | | Specific | Incubation | Method | Ref. |
| sigma 1 (h) | human | [³H] | 15 | nM | 16 | nM | haloperidol | 120 min | Scintillation | A |
| (agonist | endogenous | (+)pentazocine | | | | | (10 μM) | 37° C. | counting | |
| radioligand) | (Jurkat | | | | | | | | | |
| | cells) | | | | | | | | | |
| sigma 2 (h) | human | [3H] DTG | 25 | nM | 80.84 | nM | Haloperidol | 60 min | Scintillation | B |
| (agonist | endogenous | (+1 μM (+) | | | | | | RT | counting | |
| radioligand) | (Jurkat | Pentazocine) | | | | | (10 μM) | | | |
| | cells) | | | | | | | | | |
| dopamine | human | [³H]BTCP | 4 | nM | 4.5 | nM | BTCP | 120 min | Scintillation | C |
| transporter | recombinant | | | | | | (10 μM) | 4° C. | counting | |
| (h) | (CHO cells) | | | | | | | | | |
| (antagonist | | | | | | | | | | |
| radioligand) | | | | | | | | | | |

References for Table A: A = Pristupa, Z. B. et al. (1994), Mol. Pharmacol., 45: 125-135; B = Ganapathy, M. E. et al. (1999), J. Pharmacol. Exp. Ther., 289: 251-260; C = GANAPATHY M E. Et al. (1999), JPET, 289: 251-260.

The results are expressed as a percent of control specific binding: [(measured specific binding)/(control specific binding)]*100 and as a percent inhibition of control specific binding: 100−{[(measured specific binding)/(control specific binding)]*100}, obtained in the presence of the test compounds.

The IC₅₀ values (concentration causing a half-maximal inhibition of control specific binding) and Hill coefficients (nH) were determined by non-linear regression analysis of the competition curves generated with mean replicate values using Hill equation curve fitting: Y={D+[(A−D)/(1+(C/C₅₀)^{nH})]} (where Y=specific binding, A=left asymptote of the curve, D=right asymptote of the curve, C=compound concentration, C50=IC₅₀, and nH=slope factor). This analysis was performed using software developed at Cerep (Hill software) and validated by comparison with data generated by the commercial software SigmaPlot® 4.0 for Windows® (© 1997 by SPSS Inc.). The inhibition constants (Ki) were calculated using the Cheng Prusoff equation: Ki=(IC₅₀)/(1+L/K_{D}) (where L=concentration of ligand in the assay, and KD=affinity of the ligand for the receptor).

### Compounds Tested:

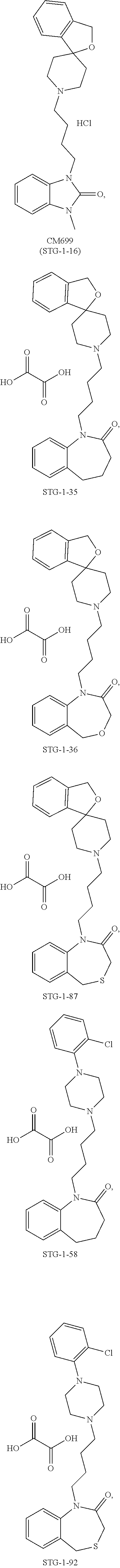

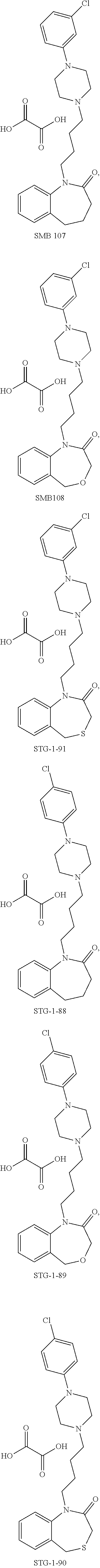

### Results:

TABLE R1
Sigma 1 Receptor

| | | % Inhibition of Control | | |
|---|---|---|---|---|
| | Concentration | Specific Binding | | |
| Compound | (M) | 1^{st} | 2^{nd} | 3^{rd} |
| CM699 | 1.00E−06 | 95.6 | 94 | 94.8 |
| CM699 | 1.00E−06 | 95.6 | 94 | 94.8 |
| STG135 | 1.00E−06 | 97.9 | 94.5 | 96.2 |
| STG136 | 1.00E−06 | 98.8 | 98.4 | 98.6 |
| STG187 | 1.00E−06 | 93.6 | 98.9 | 96.3 |
| STG158 | 1.00E−06 | 95.7 | 93.8 | 94.7 |
| STG192 | 1.00E−06 | 53.5 | 53.2 | 53.3 |
| SMB107 | 1.00E−06 | 96.7 | 97.6 | 97.1 |
| SMB108 | 1.00E−06 | 93.8 | 95.4 | 94.6 |
| STG191 | 1.00E−06 | 98.6 | 98.5 | 98.6 |
| STG188 | 1.00E−06 | 98.8 | 99.2 | 99 |
| STG189 | 1.00E−06 | 92.8 | 93.6 | 93.2 |
| STG190 | 1.00E−06 | 101.1 | 100.9 | 101 |

TABLE R2
Sigma 2 Receptor

| | | % Inhibition of Control | | |
|---|---|---|---|---|
| | Concentration | Specific Binding | | |
| Compound | (M) | 1^{st} | 2^{nd} | 3^{rd} |
| CM699 | 1.00E−06 | 88 | 95.9 | 92 |
| STG135 | 1.00E−06 | 106 | 84.4 | 95.2 |
| STG136 | 1.00E−06 | 90.3 | 100.3 | 95.3 |
| STG187 | 1.00E−06 | 96.5 | 108.3 | 102.4 |
| STG158 | 1.00E−06 | 93.2 | 100.2 | 96.7 |
| STG192 | 1.00E−06 | 56.6 | 43.4 | 50 |
| SMB107 | 1.00E−06 | 104.2 | 98.7 | 101.4 |
| SMB108 | 1.00E−06 | 95 | 99.8 | 97.4 |
| STG191 | 1.00E−06 | 86.2 | 102.5 | 94.4 |
| STG188 | 1.00E−06 | 82.9 | 85.9 | 84.4 |
| STG189 | 1.00E−06 | 82.2 | 78.2 | 80.2 |
| STG190 | 1.00E−06 | 96.4 | 95.5 | 95.9 |

TABLE R3
Dopamine Transporter

| | | % Inhibition of Control | | |
|---|---|---|---|---|
| | Concentration | Specific Binding | | |
| Compound | (M) | 1^{st} | 2^{nd} | 3^{rd} |
| CM699 | 1.00E−06 | 18.6 | 23.2 | 20.9 |
| STG135 | 1.00E−06 | 6 | 3.5 | 4.8 |
| STG136 | 1.00E−06 | 7.9 | 11.4 | 9.7 |
| STG187 | 1.00E−06 | 12.9 | 13.1 | 13 |
| STG158 | 1.00E−06 | −5.6 | −2.8 | −4.2 |
| STG192 | 1.00E−06 | 12.6 | 4.3 | 8.5 |
| SMB107 | 1.00E−06 | 24.6 | 24.4 | 24.5 |
| SMB108 | 1.00E−06 | 28.3 | 25.9 | 27.1 |
| STG191 | 1.00E−06 | 21.8 | 27.6 | 24.7 |
| STG188 | 1.00E−06 | 26.5 | 23.2 | 24.8 |
| STG189 | 1.00E−06 | 8.6 | 5.1 | 6.8 |
| STG190 | 1.00E−06 | 9.2 | 14.8 | 12 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 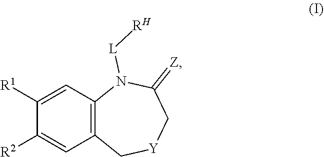
or pharmaceutically acceptable salt thereof, wherein:
Z is O, S, NR^{N}, or C(R^{C})₂;
each R^{N} is independently hydrogen, C₁₋₆ alkyl, or a nitrogen protecting group;
each R^{C} is independently hydrogen or C₁₋₆ alkyl;
Y is O, S, NR^{N}, or C(R^{C})₂;
R¹ hydrogen, halogen, substituted or unsubstituted C₁₋₁₂ alkyl;
R² hydrogen, halogen, substituted or unsubstituted C₁₋₁₂ alkyl;
L is substituted or unsubstituted C₁₋₁₂ alkylene or substituted or unsubstituted hetero-C₁₋₁₂-alkylene;
R^{H} is selected from the group consisting of: 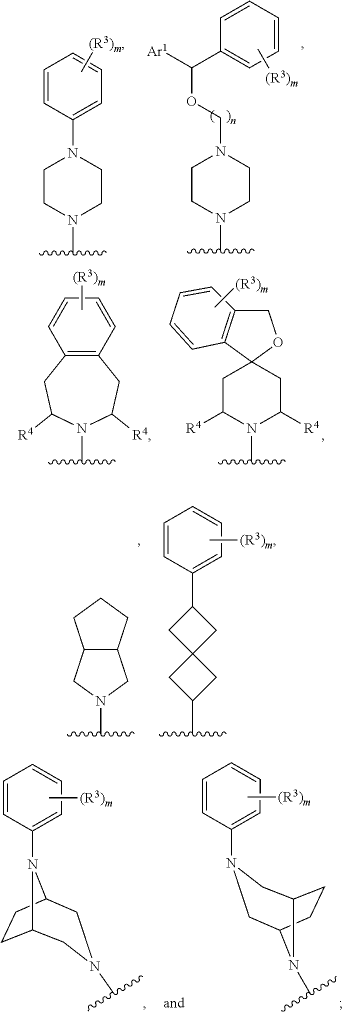
Ar¹ is substituted or unsubstituted aryl, or substituted or unsubstituted heteroaryl;
n is an integer selected from 1, 2, 3, 4, and 5;
each R³ is independently selected from the group consisting of hydrogen, halogen, —NO₂, —CN, —C(═O)R^{C}, and —C(═O)OR^{C};
each R⁴ is independently hydrogen or C₁₋₆ alkyl; and
m is an integer selected from 0, 1, 2, 3, 4, and 5.

2. The compound of claim 1, or pharmaceutically acceptable salt thereof, wherein Z is O.

3. The compound of claim 1 or 2, or pharmaceutically acceptable salt thereof, wherein Y is O.

4. The compound of claim 1 or 2, or pharmaceutically acceptable salt thereof, wherein Y is CH₂.

5. The compound of any one of claims 1-4, or pharmaceutically acceptable salt thereof, wherein R¹ is hydrogen, bromo, fluoro, or substituted or unsubstituted C₁₋₈ alkyl.

6. The compound of any one of claims 1-5, or pharmaceutically acceptable salt thereof, wherein R¹ is hydrogen.

7. The compound of any one of claims 1-6, or pharmaceutically acceptable salt thereof, wherein R² is hydrogen, bromo, fluoro, or substituted or unsubstituted C₁₋₈ alkyl.

8. The compound of any one of claims 1-7, or pharmaceutically acceptable salt thereof, wherein R² is hydrogen.

9. The compound of any one of claims 1-8, or pharmaceutically acceptable salt thereof, wherein R^{H} is selected from the group consisting of:

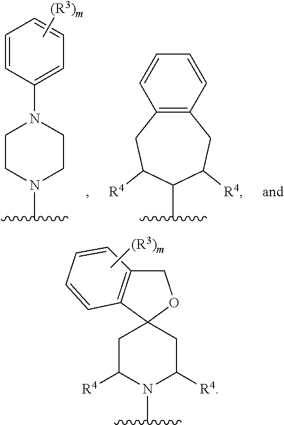

10. The compound of any one of claims 1-9, or pharmaceutically acceptable salt thereof, wherein R^{H} is:

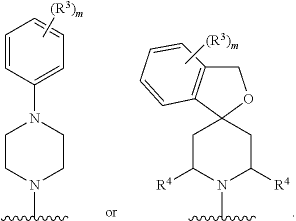

11. The compound of any one of claims 1-10, or pharmaceutically acceptable salt thereof, wherein R^{H} is:

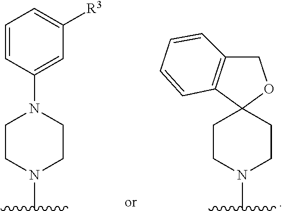

12. The compound of any one of claims 1-8, or pharmaceutically acceptable salt thereof, wherein Ar¹ is phenyl or thiopheneyl.

13. The compound of any one of claims 1-8 and 12, or pharmaceutically acceptable salt thereof, wherein Ar¹ is

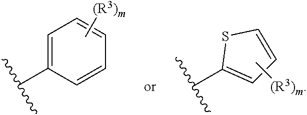

14. The compound of any one of claims 1-10 and 12-13, or pharmaceutically acceptable salt thereof, wherein m is 1.

15. The compound of any one of claims 1-10 and 12-13, or pharmaceutically acceptable salt thereof, wherein m is 0.

16. The compound of any one of claims 1-15, or pharmaceutically acceptable salt thereof, wherein L is C₁₋₆ alkylene.

17. The compound of any one of claims 1-16, or pharmaceutically acceptable salt thereof, wherein L is C₄ alkylene.

18. The compound of any one of claims 1-15, or pharmaceutically acceptable salt thereof, wherein L is hetero-C₁₋₆-alkylene.

19. The compound of any one of claims 1-15 and 16, or pharmaceutically acceptable salt thereof, wherein L is

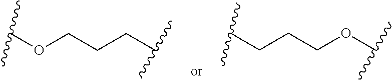 [SMILES: *CCCO* ; *CCCO*]

20. The compound of any one of claims 1-3 and 5-17, or pharmaceutically acceptable salt thereof, wherein the compound is of Formula (IA), (IB), or (IC): 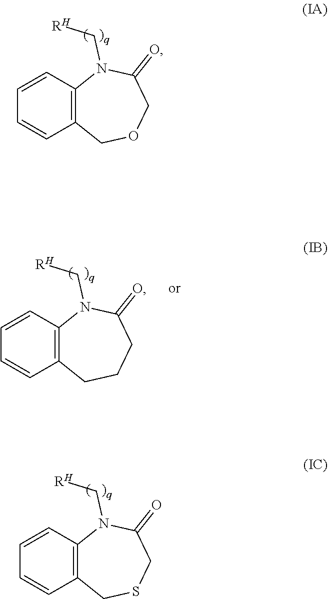
wherein q is an integer selected from 1, 2, 3, 4, 5, or 6.

21. The compound of any one of claims 1-2 and 4-17, or pharmaceutically acceptable salt thereof, wherein the compound is of Formula (IA-1), (IA-2), (IB-1), (IB-2), (IC-1), or (IC-2): 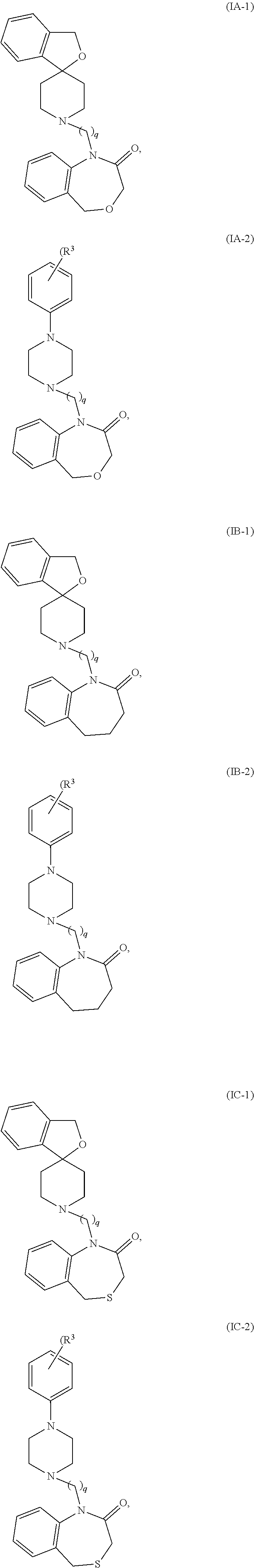
wherein q is an integer selected from 1, 2, 3, 4, 5, or 6.

22. The compound of claim 1, or pharmaceutically acceptable salt thereof, wherein the compound is of the formula:

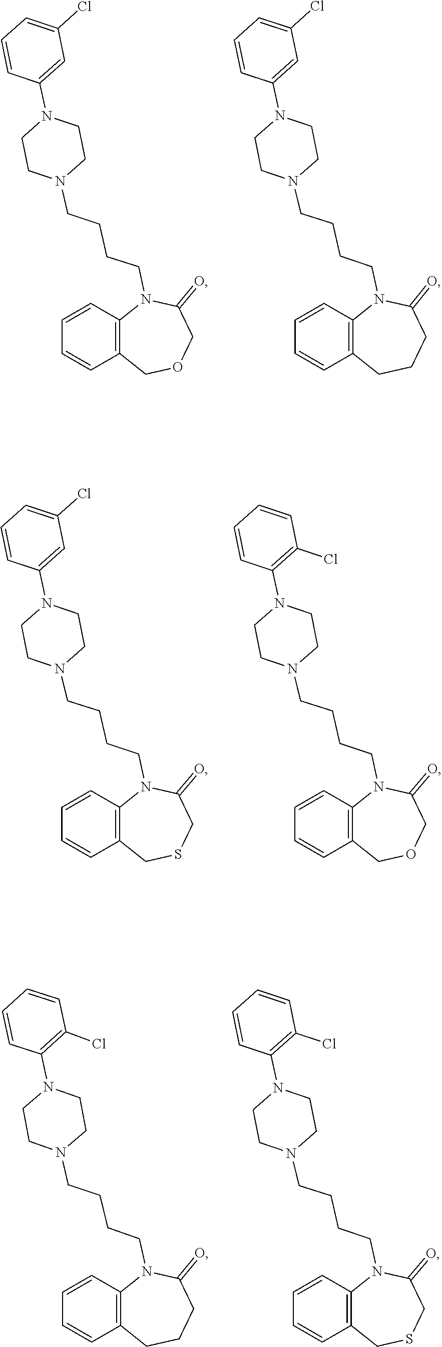 [SMILES: O=C1COCc2ccccc2N1CCCCN1CCN(c2cccc(Cl)c2)CC1 ; O=C1CCCc2ccccc2N1CCCCN1CCN(c2cccc(Cl)c2)CC1 ; O=C1CSCc2ccccc2N1CCCCN1CCN(c2cccc(Cl)c2)CC1 ; O=C1COCc2ccccc2N1CCCCN1CCN(c2ccccc2Cl)CC1 ; O=C1CCCc2ccccc2N1CCCCN1CCN(c2ccccc2Cl)CC1 ; O=C1CSCc2ccccc2N1CCCCN1CCN(c2ccccc2Cl)CC1]

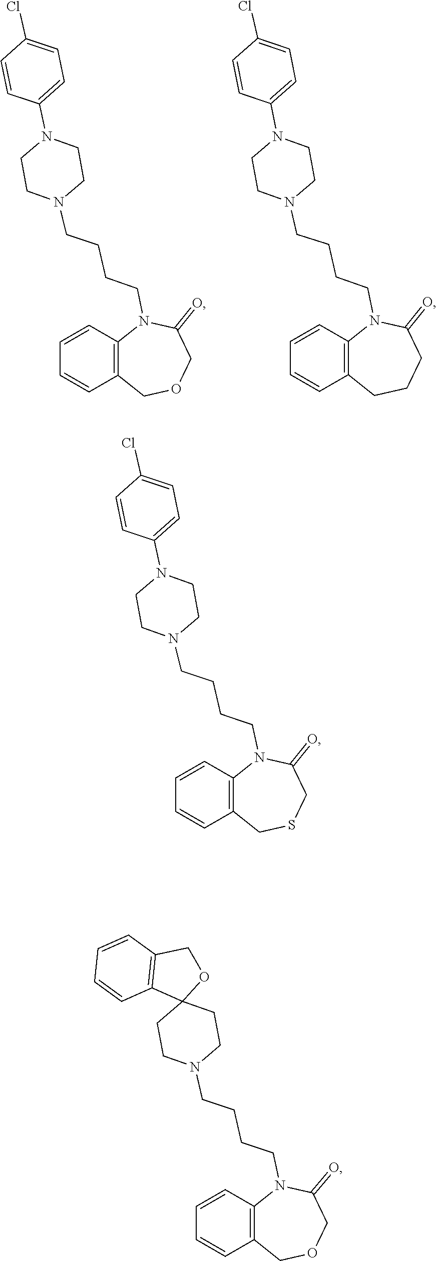 [SMILES: O=C1COCc2ccccc2N1CCCCN1CCN(c2ccc(Cl)cc2)CC1 ; O=C1CCCc2ccccc2N1CCCCN1CCN(c2ccc(Cl)cc2)CC1 ; O=C1CSCc2ccccc2N1CCCCN1CCN(c2ccc(Cl)cc2)CC1 ; O=C1COCc2ccccc2N1CCCCN1CCC2(CC1)OCc1ccccc12]

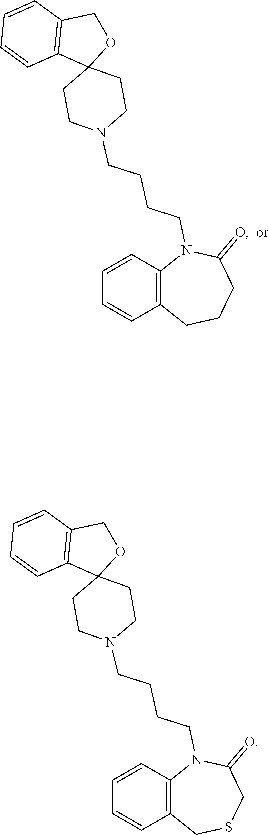

23. The compound of any one of claims 1-22, or pharmaceutically acceptable salt thereof, wherein the compound is sigma-receptor antagonist.

24. The compound of any one of claims 1-22, or pharmaceutically acceptable salt thereof, wherein the compound is dopamine transporter inhibitor.

25. A composition comprising the compound of any one of claims 1-24, or pharmaceutically acceptable salt thereof, and a pharmaceutically acceptable excipient.

26. A method of treating or preventing substance intake by a subject comprising administering to the subject a therapeutically effective amount of the compound of any one of claims 1-24, or pharmaceutically acceptable salt thereof, or composition of claim 25.

27. A method of treating or preventing substance use disorder in a subject comprising administering to the subject a therapeutically effective amount of the compound of any one of claims 1-24, or pharmaceutically acceptable salt thereof, or composition of claim 25.

28. A method of treating the symptoms of substance use disorder in a subject comprising administering to the subject a therapeutically effective amount of the compound of any one of claims 1-24, or pharmaceutically acceptable salt thereof, or composition of claim 25.

29. A method of treating or preventing substance addiction in a subject comprising administering to the subject a therapeutically effective amount of the compound of any one of claims 1-24, or pharmaceutically acceptable salt thereof, or composition of claim 25.

30. A method of treating the symptoms of substance addiction in a subject comprising administering to the subject a therapeutically effective amount of the compound of any one of claims 1-24, or pharmaceutically acceptable salt thereof, or composition of claim 25.

31. The method of any one of claims 26-30, wherein the drug or stimulant is methamphetamine.

32. The method of any one of claims 26-30, wherein the drug or stimulant is cocaine.

33. A method of treating or preventing neurotoxic effects resulting from substance use disorder, substance addiction, and/or substance intake by a subject comprising administering to the subject a therapeutically effective amount of the compound of any one of claims 1-24, or pharmaceutically acceptable salt thereof, or composition of claim 25.

34. A method of treating or preventing a disease or disorder associated with one or more sigma receptors comprising administering to a subject a therapeutically effective amount of the compound of any one of claims 1-24, or pharmaceutically acceptable salt thereof, or composition of claim 25.

35. A method of treating or preventing a disease or disorder associated with one or more dopamine transporters comprising administering to a subject a therapeutically effective amount of the compound of any one of claims 1-24, or pharmaceutically acceptable salt thereof, or composition of claim 25.

36. A method of treating or preventing a disease or disorder associated with one or more dopamine transporters and with one or more sigma receptors comprising administering to a subject a therapeutically effective amount of the compound of any one of claims 1-24, or pharmaceutically acceptable salt thereof, or composition of claim 25.

37. A kit comprising:
the compound of any one of claims 1-24, or pharmaceutically acceptable salt thereof, or composition of claim 25; and
and instructions for administering the compound, or pharmaceutically acceptable salt thereof, or composition to a subject.
